(19) United States
(12) Patent Application Publication
(10) Pub. No.: US 2023/0263632 A1
(43) Pub. Date: Aug. 24, 2023

(54) ANCHOR FOR PROSTHETIC CARDIAC VALVE DEVICES

(71) Applicant: SHIFAMED HOLDINGS, LLC, Campbell, CA (US)

(72) Inventors: Alice YANG, Campbell, CA (US); Connor MULCAHY, San Francisco, CA (US); Jordan SKARO, San Jose, CA (US); Jonathan OAKDEN, San Jose, CA (US); Jasper Ellington ADAMEK-BOWERS, San Francisco, CA (US); Andrew BACKUS, Santa Cruz, CA (US); Brian J. FAHEY, Palo Alto, CA (US)

(21) Appl. No.: 18/043,542

(22) PCT Filed: Aug. 27, 2021
(86) PCT No.: PCT/US2021/047851

§ 371 Date: Feb. 28, 2023

Related U.S. Application Data
(60) Provisional application No. 63/072,853, filed on Aug. 31, 2020.
(60) Provisional application No. 63/141,412, filed on Jan. 25, 2021.

(51) Int. Cl. A61F 2/24

(52) U.S. Cl. CPC A61F 2/2457; A61F 2/2418; A61F 2230/0091; A61F 2250/0032

(57) ABSTRACT

Devices, systems, and methods related to heart valve prostheses. The prosthesis includes an anchor and a frame. The anchor is shaped to encircle chordae and/or leaflets of a native heart valve. The frame is configured to sit within the anchor and the native valve such that the anchor encircles and anchors the frame in place. At least a portion of the anchor is configured to be visible using surgical visualization techniques during delivery of the prosthesis within the heart. The anchor can include an echogenic portion configured to enable viewing of the echogenic portion with ultrasound during delivery of the prosthesis.

### CROSS REFERENCE TO RELATED APPLICATIONS

This application claims priority to U.S. Provisional Application No. 63/072,853, titled “Anchor for Prosthetic Cardiac Valve Delivery Devices and Systems,” filed Aug. 31, 2020, and to U.S. Provisional Application No. 63/141,412, titled “Anchor For Prosthetic Cardiac Valve Devices,” filed Jan. 25, 2021, the entireties of which are incorporated by reference herein.

### BACKGROUND

Blood flow between heart chambers is regulated by native valves - the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient’s lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transseptally, requiring transapical access instead. Furthermore, existing mitral valve replacement devices are not optimized with respect to strength-weight ratio and often take up too much space within the valve chambers, resulting in obstruction of outflow from the ventricle into the aorta and/or thrombosis.

Thus, a new valve device that overcomes some or all of these deficiencies is desired. Further, it would be desirable for at least a portion of such valve device to be readily viewable using medical imaging during deployment of one or more portions of the valve device to assure proper positioning of the valve device.

### SUMMARY

Described herein are systems, devices, and methods related to heart valve prostheses. The prosthesis can include a frame and an anchor. The frame can include an expandable stent-like structure with a central lumen having leaflets. The frame can be configured to be placed within a diseased native valve to replace the diseased native valve. The anchor can have a spiral shape that is configured to encircle an outer perimeter of the frame. The anchor can provide an opposing force against the frame to anchor the frame with respect to native valve.

In some implementations, the anchor is deployed within the heart separately from the frame. During deployment, the anchor may extend from a catheter (e.g., as part of an anchor deployment catheter system) to transition from a substantially straight configuration to a deployed spiral-shaped configuration. Once deployed within the heart, the position of the anchor can be manipulated to wrap the anchor around the chordae tendineae and/or native valve leaflets. The anchor can include features that promote visualization of the anchor during deployment and/or positioning of the anchor within the heart. For example, the anchor may include one or more echogenic portions that enable viewing of the anchor within the heart using ultrasound imaging.

According to some aspects, a prosthesis for treating a diseased native valve comprises: a frame structure having a plurality of leaflets therein; and a spiral anchor configured to extend around an outer perimeter of the frame structure, the spiral anchor comprising an echogenic portion configured to enable viewing of the echogenic portion with ultrasound during delivery of at least a portion of the prosthesis.

In these aspects, the echogenic portion may be a distal tip of the anchor. In these aspects, the echogenic portion may comprise a wire wound around the distal tip. In these aspects, the wire may comprise stainless steel or nitinol. In these aspects, the wire may be embedded in an encapsulation layer. In these aspects, the encapsulation layer may comprise an adhesive, a laminate, or a urethane. In these aspects, the echogenic portion may be at least a portion of a grabber arm of the anchor. In these aspects, the echogenic portion may be at least a portion of a main body of the anchor. In these aspects, the echogenic portion may comprise at least 75% of a length of the anchor. In these aspects, the echogenic portion may comprise a wire wound around the main body. In these aspects, the wire may comprise stainless steel or nitinol. In these aspects, the wire may be wound in a pattern of alternating high-density portions and low-density portions. In these aspects, the high-density portion may be wound at a pitch of less than 1 mm and the low-density portion is wound at a pitch of greater than 1 mm. In these aspects, a distal tip of the anchor may further comprise a second echogenic portion, the echogenic portion of the main body and the second echogenic portion spaced apart by a non-echogenic portion. In these aspects, the spiral anchor may comprise a grabber arm at a distal end thereof, the grabber arm extending radially outwards and having a portion of lower curvature than the main body, further wherein the grabber arm comprises the non-echogenic portion. In these aspects, the echogenic portion may comprise a plurality of markers configured to indicate a planarity of the spiral anchor when viewed with ultrasound. In these aspects, the echogenic portion may comprise a plurality of echogenic sections having varying lengths or varying spacing along the main body. In these aspects, the echogenic portion may comprise a plurality of echogenic bands along a portion of the anchor, wherein spaces between the echogenic bands are predetermined and configured to determine an angular position of the anchor when viewed under ultrasound. In these aspects, the echogenic portion may comprise a plurality of reflective elements configured to reflect ultrasound waves. In these aspects, each of the reflective elements may include an arrangement of planar reflective surfaces that form an inset. In these aspects, the reflective elements may extend around a circumference of the echogenic portion. In these aspects, the echogenic portion may be made of a metallic material or alloy. In these aspects, the metal material may include a stainless steel, a nickel titanium alloy, or a stainless steel and a nickel titanium alloy. In these aspects, the echogenic portion may be distal to a grabber arm of the anchor, the grabber arm extending radially outwards from a main body of the anchor and comprising a lower curvature than the main body. In these aspects, the echogenic portion may have a curvature in the same direction as a curvature of a main body of the anchor. In these aspects, the echogenic portion may comprise a same material as a main body of the anchor. In these aspects, the main body of the anchor may be a wire made of a shape memory material. In these aspects, the echogenic portion may be a distal tip of the anchor and has a length ranging between 2 mm and 10 mm. In these aspects, the echogenic portion may be a distal tip of the anchor and have a diameter greater than a diameter of a main body of the anchor. In these aspects, the echogenic portion may be a distal tip of the anchor and has a diameter ranging between 2 mm and 4 mm.

According to some aspects, a method of delivering an anchor of a valve prosthesis comprises: advancing a distal end of a delivery device to a first side of a native valve; deploying an anchor from a delivery configuration to a deployed configuration on the first side of the native valve, the anchor comprising an echogenic portion; advancing the anchor in the deployed configuration from the first side of the native valve to a second side of the native valve; rotating the anchor in the deployed configuration around one or more structures on the second side of the native valve; identifying the echogenic portion in an ultrasound image to confirm that the anchor has been fully rotated around the one or more structures; and releasing the anchor from the distal end of the delivery device.

In these aspects, the method may further comprise identifying the echogenic portion with ultrasound during the rotating step. In these aspects, the method may further comprise identifying the echogenic portion with ultrasound during the advancing step. In these aspects, identify the echogenic portion in the ultrasound further may comprise confirming a planarity of the anchor. In these aspects, the anchor may comprise any of the anchors of described herein. In these aspects, the method may further comprise deploying a frame of the valve prosthesis within the native valve and within the anchor. In these aspects, the method may further comprise capturing the one or more structures on the second side of the heart using with an outwardly radially extending grabber arm of the anchor. In these aspects, the anchor may have a spiral shape. In these aspects, the echogenic portion may be at least a distal tip of the anchor, wherein identifying the echogenic portion may include identifying a location of at least the distal tip of the anchor. In these aspects, the echogenic portion may be at least a portion of a main body of the anchor, the main body having a spiral shape, wherein identifying the echogenic portion may include identifying a location of at least the portion of the main body of the anchor. In these aspects, the echogenic portion may at least a portion of an outwardly radially extending grabber arm of the anchor, wherein identifying the echogenic portion may include identifying a location of at least the portion of the grabber arm of the anchor. In these aspects, the echogenic portion may comprise a wire wound around the distal tip. In these aspects, the echogenic portion may comprise reflective elements each having an arrangement of reflective surfaces that reflect ultrasound. In these aspects, the method may further comprise releasing the anchor from the delivery device after anchor placement is confirmed.

According to some aspects, an anchor for anchoring a frame portion of valve prosthesis comprises: a spiral-shaped main body configured to extend around one or more structures of a native valve; and an echogenic portion configured to enable viewing of the anchor with ultrasound during delivery of the anchor around the one or more structures of the native valve.

In these aspects, the echogenic portion may be a distal tip of the anchor. In these aspects, the echogenic portion may include at least a portion of the main body of the anchor. In these aspects, the anchor may further comprise a grabber arm between the main body and a distal tip of the anchor, wherein the echogenic portion includes at least a portion of the grabber arm. In these aspects, the grabber arm may extend radially outwards with respect to the main body. In these aspects, the anchor may further comprise a grabber arm between the main body and a distal tip of the anchor, wherein the echogenic portion is not part of the grabber arm. In these aspects, the echogenic portion may comprise a wire wound around a distal tip of the anchor. In these aspects, the echogenic portion may comprise a wire wound around the main body of the anchor. In these aspects, the echogenic portion may comprise a plurality of echogenic bands along a main body, a grabber arm, or the main body and the grabber arm of the anchor. In these aspects, spaces between the echogenic bands may be predetermined and configured to determine an angular position of the anchor when viewed under ultrasound. In these aspects, the echogenic portion may have a curvature in the same direction as a curvature of the main body of the anchor. In these aspects, the echogenic portion may comprise a plurality of reflective elements configured to reflect ultrasound waves. In these aspects, each of the reflective elements may include an arrangement of planar reflective surfaces that form an inset. In these aspects, the echogenic portion may have a curvature in a same direction as a curvature of the main body of the anchor. In these aspects, the reflective elements may extend around a diameter of the echogenic portion. In these aspects, the reflective elements may be arranged in a repeated pattern along the echogenic portion. In these aspects, the echogenic portion may be made of a metal material. In these aspects, the metal material may include one or more of stainless steel and nitinol. In these aspects, the anchor may further comprise a non-conductive or non-metallic outer covering over at least the echogenic portion.

These and other aspects are described herein.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIGS. 1A and 1B illustrate an exemplary valve prosthesis including a frame and an anchor: FIG. 1A shows a side view of the valve prosthesis; and FIG. 1B shows a top view of the valve prosthesis.

FIGS. 2A-2H illustrate sequential views of an exemplary method of implanting a valve prosthesis.

FIGS. 3A-3C illustrate an exemplary anchor having an echogenic distal tip having a coil: FIG. 3A shows a section view of the echogenic distal tip; FIG. 3B shows a close-up perspective view of the echogenic distal tip; and FIG. 3C shows a perspective view of the anchor.

FIGS. 4A and 4B illustrate exemplary ultrasound images of anchors during deployment: FIG. 4A shows an anchor without a coiled echogenic tip; and FIG. 4B shows an anchor with a coiled echogenic tip.

FIG. 5 illustrates an exemplary anchor having a grabber arm extending from a main body of the anchor.

FIGS. 6A and 6B illustrate two exemplary anchors having grabber arms of different lengths.

FIGS. 7A and 7B illustrate an exemplary anchor having a coiled echogenic distal tip and echogenic main body.

FIG. 8 illustrates an exemplary variation of the anchor of FIGS. 7A and 7B, where the echogenic main body include echogenic markers (e.g., bands).

FIG. 9 illustrates an exemplary echogenic portion having reflective features for reflecting ultrasound waves.

FIGS. 10A-10C illustrates an exemplary variation of the echogenic portion of FIG. 9, where the echogenic portion has a curved geometry: FIG. 10A shows a side view of the echogenic portion; FIG. 10B shows a front view of the echogenic portion; and FIG. 10C shows a top view of an anchor with the echogenic portion at a distal end of the anchor.

FIG. 11 is a flowchart illustrating an exemplary method of delivering an anchor and valve prothesis into a patient’s heart.

### DETAILED DESCRIPTION

Described herein are systems, devices, or methods for treatment or replacement of a diseased native valve of the heart, for example a mitral valve.

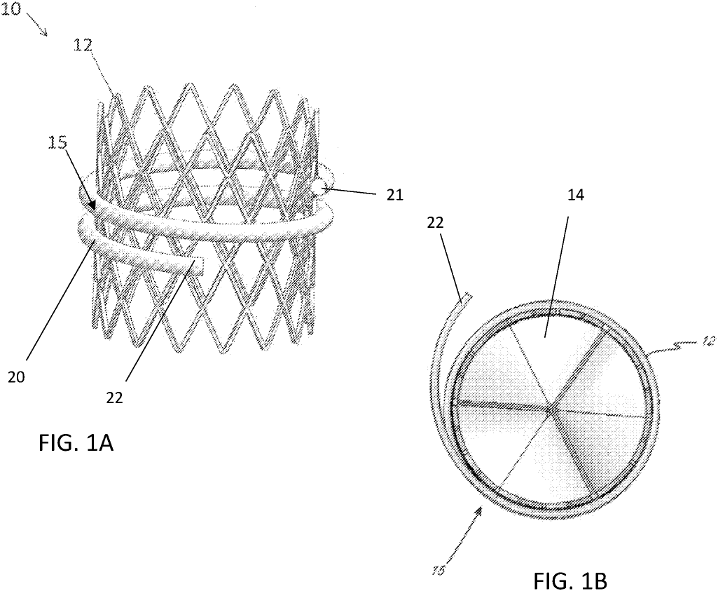

FIGS. 1A-1B show an exemplary valve prosthesis 10 (also referred to herein as “valve device”) for replacement of a valve, such as a mitral valve. The illustrated valve prosthesis 10 comprises a frame structure 12, leaflets 14, and an anchor 15. The anchor 15 includes a wire 20 formed in a spiral shape around the frame structure 12.

The exemplary frame structure 12 is configured like a stent. The frame structure 12 has an expanded state and an unexpanded (e.g., collapsed or compressed) state. The compressed state is sized and dimensioned for percutaneous insertion and the expanded state is sized and dimensioned for implantation in a native valve of a patient, such as a mitral valve.

The anchor 15 can include a spiral member, such as wire 20, having a proximal end 21 and a distal end 22. The anchor 15 can be configured to engage with the frame structure 12 via a compression fit. The wire 20 can be formed of a material having sufficient rigidity to hold a predetermined shape. In an exemplary embodiment, the wire 20 can be formed of a shape memory material (e.g., nitinol). Further, the anchor 15 prior to implantation may comprise a flat spiral shape such that loops of the anchor are generally positioned within the same plane (the plane being perpendicular to a longitudinal axis of a delivery device). Additionally, in some embodiments, the distal end 22 can be rounded and/or atraumatic.

The valve prosthesis 10 can be configured for replacing a mitral valve with the distal end 22 configured for insertion through a commissure.

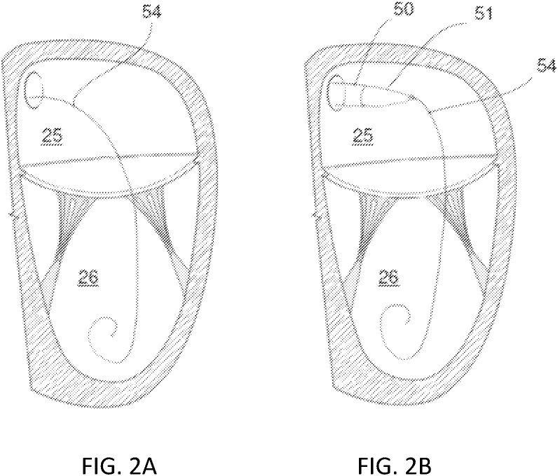

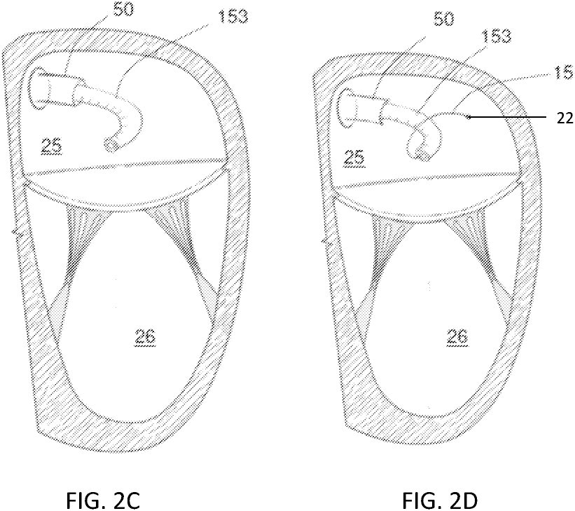

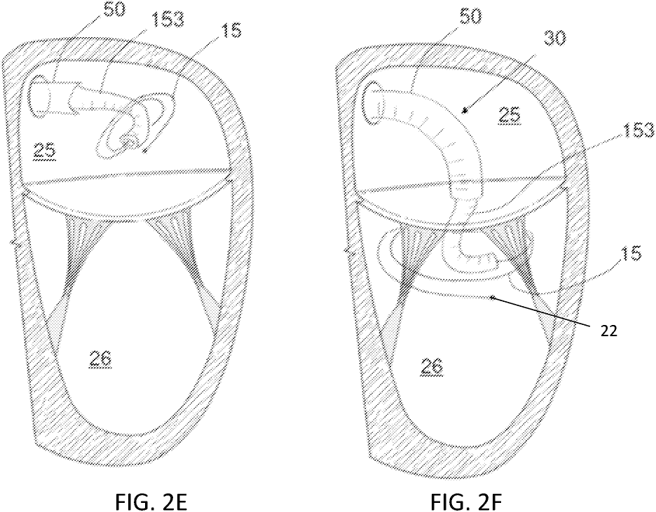

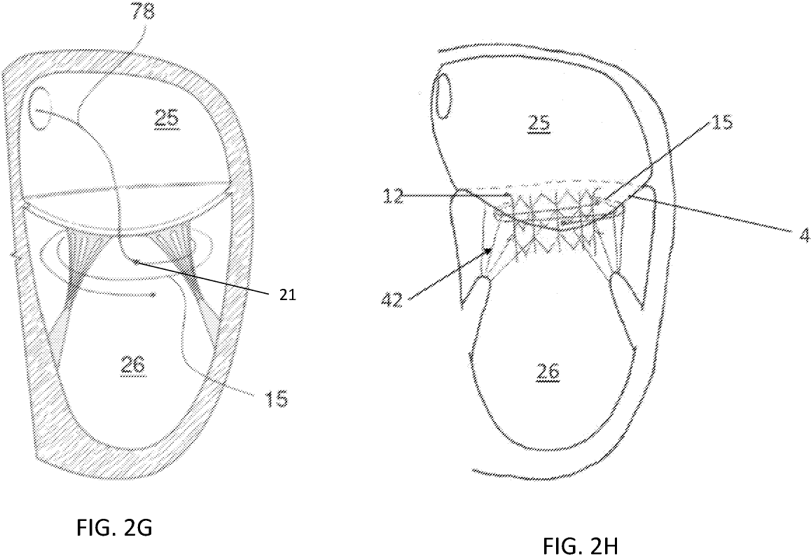

FIGS. 2A-2H show sequential views of an exemplary method of implanting a valve prosthesis 10. At FIG. 2A, a transseptal puncture is made. A guidewire 54 is then routed through the puncture site and left either in the left atrium 25 or across the mitral valve into the left ventricle 26. At FIG. 2B, the outer sheath 50 (optionally with an inner dilator 51) is tracked over the guidewire 54 until the distal end of the outer sheath 50 protrudes into the left atrium 25. The guidewire 54 and inner dilator 51 are then removed from the outer sheath 50. At FIG. 2C, an inner shaft and attached distal anchor guide 153 are inserted through the outer sheath 50 until the distal tip of the anchor guide 153 extends into the left atrium 25. The anchor guide 153 can be positioned and/or oriented as desired by steering the distal end of the sheath 50 and/or rotating the inner shaft and anchor guide 153 relative to the sheath 50. At FIG. 2D, once the anchor guide 153 is in the correct orientation, the anchor 15 can be pushed out through distal tip of the anchor guide 153 (with the distal tip 22 extending out of the guide 153 first). At FIG. 2E, the anchor 15 can fully deploy into the atrium 25. At FIG. 2F, the entire delivery system 30 can be pushed and steered (for example, via steering mechanisms in the outer sheath 50) towards an apex of the ventricle 26, crossing through the mitral valve. In some embodiments, counter-rotation of the anchor 15 may aid in advancing the anchor 15 across the mitral valve without tangling. Once the anchor 15 is at the correct depth within the ventricle 26, forward rotation of the anchor 15 (via forward rotation of the inner shaft and guide 153) will promote encircling of the mitral leaflets and chordae by the anchor 15 (i.e., with the distal end 22 leading the encircling). At FIG. 2G, the outer sheath 40, inner sheath, and anchor guide 153 are removed, leaving a tether 78 in place (and attached to the proximal end 21 of the anchor 15). At FIG. 2H, the frame structure 12 can then be delivered over the tether 78 and into place within the anchor 15. The tether 78 can then be released from the proximal end 21 of the anchor 15 to leave the prosthesis 10 in place in the mitral valve 4.

In some examples, the anchor 15 and the frame structure 12 are delivered into the heart using different catheter systems. One or more catheter systems can include nested catheters. For example, the anchor 15 may be delivered and deployed within the heart using an anchor delivery catheter system, which may include the outer sheath 50 and the anchor guide 153. The frame structure 12 may be delivered and deployed within the heart using a valve deployment catheter system having one or more catheters. The various catheters of the anchor delivery catheter system and the valve delivery catheter system may travel over the tether 15. Exemplary methods and devices for delivering an anchor/valve prosthesis are described in U.S. Pat. Application No. 16/824,576, filed Mar. 19, 2020, published as U.S. Pat. Application Publication No. US20200297491A1, and U.S. Pat. Application No. 16/594,946, filed Oct. 7, 2019, issued as U.S. Pat. No. 10/912,644, the entireties of which are incorporated by reference herein.

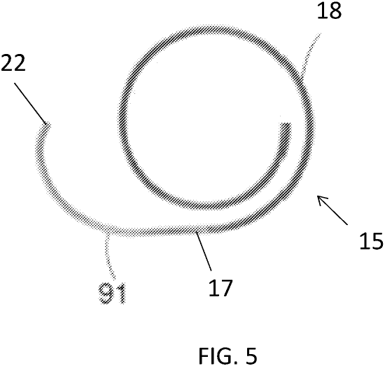

The distal end 22 of the anchor 15 described herein can be formed as part of a “grabber” arm. The grabber arm can be configured to extend radially outwards from the rest of the anchor 15 (e.g., at least a main body of the anchor 15) to help capture one or more native valve structures (e.g., the chordae) as the anchor 15 is rotated during delivery. For example, FIG. 5 shows an exemplary anchor 15 having a main body 18 with a grabber arm 91 extending therefrom. The grabber arm 91 can be continuous with the anchor 15 and can spiral in the same direction (e.g., clockwise or counterclockwise). The grabber arm 91 can include one or more portions that have different curvatures than the curvature of a main body 18. As shown in FIG. 5, the grabber arm 91 includes a generally straight portion 17 that is proximal to an arcuate portion that includes the distal end 22. One or more portions of (e.g., relatively) lower curvature on the grabber arm cause the distal end 22 to extend in a wider arc or angle than the main body 18 of the anchor 15, thereby providing a larger space with which to grab the structures (e.g., chordae) of the native valve. In some embodiments, the distal tip 22 of the grabber arm 91 can have a portion of relatively greater curvature than that of the main body 18. Greater curvature can cause the distal tip 22 to hook or bend radially inwards to further aid in grabbing of native valve structure. Once implanted, the grabber arm 91 can be shaped and sized so as not to engage or anchor against native valve structures (while the main body 18 conversely does engage with the native valve structures). The grabber arm 91 can include an echogenic tip thereon to assist with visualization during capture of the native valve structures and/or implantation of the prosthetic valve, as described further below.

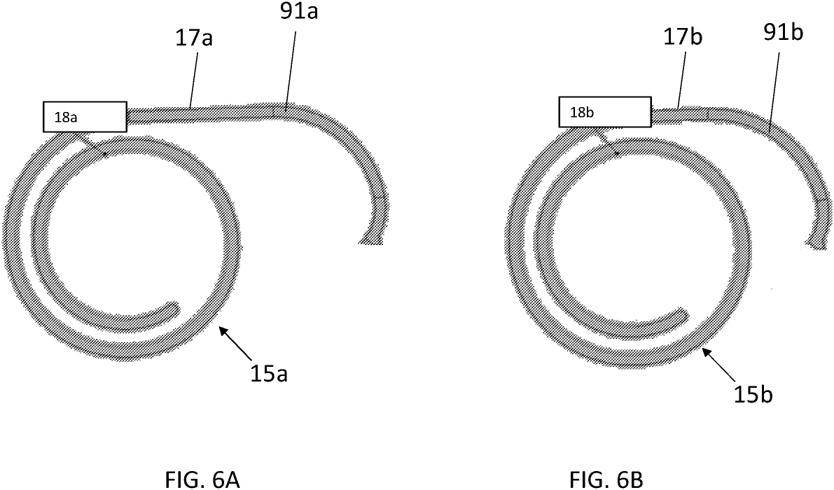

Referring to FIGS. 6A-6B, the anchor 15a,15b can advantageously be sized to accommodate different annular diameters by only changing the length or size of the grabber arm 91 while keeping the diameter of the spiral constant between sizes. In some embodiments, the length of a straight portion can be varied to accommodate different annular diameters, while the remainder of the anchor remains the same. For example, the anchor 15a shown in FIG. 6A can be configured to fit in a 55 mm diameter native annulus while the anchor 15b shown in FIG. 6B can be configured to fit in a 40 mm diameter native annulus. The diameter and shape of the central spirals of the anchors 15a, 15b of FIGS. 6A and 6B can be equivalent. For example, the length 18a and diameter of the main body of anchor 15a can be the same as the length 18b and diameter of the main body of anchor 15b. However, the section 17a of grabber arm 91a of anchor 15a can be longer (and thus extend further radially outwards) than the section 17b grabber arm 91b of anchor 15b. As a result of the longer grabber arm 91a, the anchor 15a of FIG. 6A can accommodate (i.e., function within) a larger native mitral valve annulus than the anchor 15b of FIG. 6B.

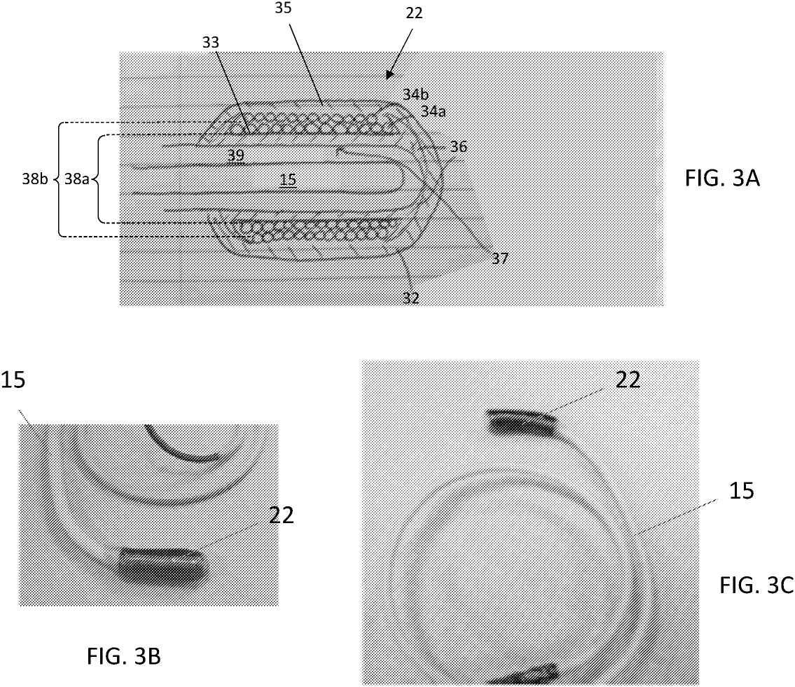

Referring to FIGS. 3A-3C, in some embodiments, the distal end 22 can include an echogenic portion (i.e., a portion configured to be bright or clearly visible under ultrasound). In one embodiment (and as shown in FIG. 3A), the echogenic portion can include a coil 33 (e.g., a wire wrapped, wound, or spiraled about the distal end 22). The coil 33 can include either a single layer positioned circumferentially around the distal end 22 or multiple layers to provide increased cross-sectional area or visibility. In the example of FIG. 3A, the wire of the coil 33 encircles the distal end 22 of the anchor 15 in two layers: a first layer 34a and a second layer 34b. In some embodiments, the first layer 34a is wrapped to have an inner diameter ranging between 0.05 inches and 0.08 inches, and the second layer 34b is wrapped to have an inner diameter ranging between 0.06 inches to 0.09 inches. In some embodiments, the diameter (gauge) of the coil 33 (wire) ranges between 0.005 inches and 0.1 inches. Further, the coil 33 can be made of any material with suitable reflectivity to ultrasound, such as stainless steel or nitinol wire.

In some embodiments, the coil 33 can also be encapsulated in one or more layers 35 of material, which can advantageously help ensure that the structure and winding of the coil 33 is maintained. The encapsulation layer(s) 35 can include, for example, an adhesive 36, a laminate 39 such as polyether block amide (PEBA), and/or an outer polymer layer 32 (e.g., implantable urethane or hydrophobic oligomer). The outer polymer layer 32 may provide a non-conductive and/or non-metallic covering to the coil 33 to prevent metal-to-metal contact with other metallic portions of the prosthesis (e.g., portions of the frame structure). In some embodiments, the coil 33 can be welded in place with or without the encapsulation layer 35. In some embodiments, the echogenic portion of the distal end 22 can include silicone foam in addition to or instead of the coil structure.

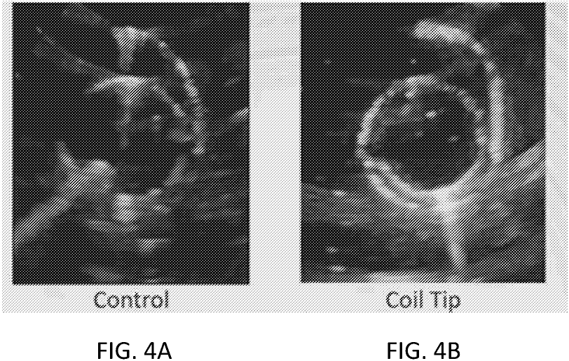

Referring to FIGS. 4A and 4B, the echogenic coil can provide enhanced visibility when an ultrasound (e.g., echocardiography) is used during deployment of the anchor 15 and encircling of the anchor 15 about the chordae. FIG. 4A shows an anchor without a coiled echogenic tip, and FIG. 4B shows an anchor with a coiled echogenic tip. As illustrated, the coiled echogenic tip (FIG. 4B) is sufficiently visible such that the distal end of the anchor is readily identifiable and locatable.

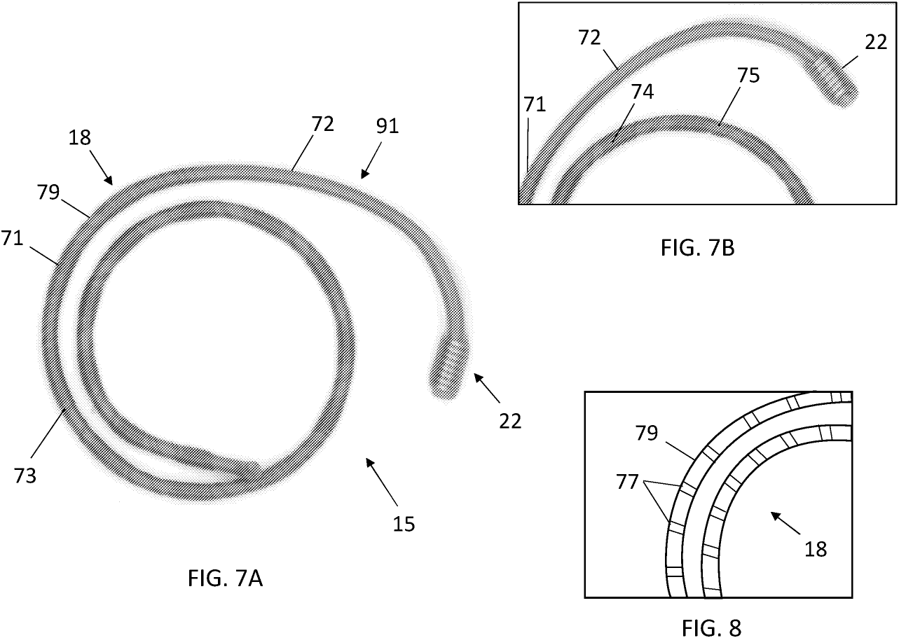

In some embodiments, the anchor includes one or more echogenic portions other than a distal tip. For example, referring to FIGS. 7A-7B, in some embodiments, at least a portion 71 of the main body 18 of the anchor 15 can be echogenic in addition to (and/or instead of) the echogenic distal tip 22. In some examples, the echogenic portion 71 of the main body 18 can extend at least 35%, at least 50%, or at least 80% of the entire length of the anchor 15. In some examples, the echogenic portion 71 of the main body 18 can extend between about 35% and 80% of the entire length of the anchor 18. In some examples, the echogenic portion 71 can include or be created by a coil 73 (e.g., made of stainless steel and/or nitinol) wrapped around a central component 79 along at least a portion of the length of the anchor 15. The coil 73 can be, for example, a wire. The central component 79 may itself include a wire, such as a memory shape wire (e.g., made of nitinol) that preferably takes on the overall spiral shape of the anchor 15 (e.g., once deployed from a delivery device). In this example, the wire/coil 73 wraps around at least a portion of the length of the main body 18. In some examples, the coil 73 may have a diameter ranging between 0.005 inches and 0.1 inches (e.g., 0.005 inches, 0.01 inches, 0.015 inches, 0.05 inches, 0.065 inches, 0.075 inches, 0.08 inches, or 0.1 inches). In some embodiments, the coil 73 can include a single wire. In some embodiments, the coil 73 includes two or more wires that are contacting one another and wound together to form the coil 73. In some embodiments, the coil 73 may be wrapped in a continuous or constant pitch along the length of the main body 18 of the anchor 15. In other embodiments, and as shown in FIGS. 7A-7B, the coil 73 can be wrapped in a pattern. The pattern can advantageously indicate the location of the main body of the anchor 15 while reducing artifacts in the image that may result if the echogenicity is too intense. For example, the pattern of echogenicity can include alternating high density portions 74 (e.g., wherein the echogenic coil is wound at a pitch of 0 mm to1 mm) and low density portions 75 (e.g., where the echogenic coil is wound at a pitch of 1 mm to8 mm). In some embodiments, the portions 74, 75 can be 1 mm to 10 mm in length, (e.g., 2 mm to5 mm in length). In some embodiments, the portions 74 and 75 are of a same (e.g., axial) length. In some embodiments, the portions 74 and 75 are of different (e.g., axial) lengths. In some embodiments, at least one discrete portion of the portions 74 and/or 75 is of a different length than a remainder of the portions 74 and 75.

The echogenic portion 71 in the main body 18 of the anchor 15 can advantageously enable viewing of the anchor 15 during delivery (e.g., while the anchor 15 is delivered to the mitral valve and encircled around the chordae) and also enable confirmation that the anchor 15 has been encircled around the chordae after completion.

In some embodiments, at least a portion of the grabber arm 91 (and/or the main body 18) may include a non-echogenic portion 72. In some embodiments, the non-echogenic portion can extend between the echogenic portion 71 and the echogenic distal end 22, which can advantageously help the user distinguish the distal end 22 from the main body 18 of the anchor 15 during delivery. In some embodiments, the non-echogenic portion 72 can correspond to the grabber arm 91 described herein. In some examples, the grabber arm 91 need not be echogenic to confirm that the anchor 15 has encircled the chordae. This may be because the grabber arm 91 may extend radially outward relative to the main body 18 after implantation rather than actively engaging with the chordae.

In some embodiments, the coil 73 can alternatively or additionally be wrapped in a pattern of varying lengths. For example, the anchor 15 can include sections that are wrapped in the coil 73 and sections that are not, and the sections can vary in length. As another more specific example, the high density portions 74 can be a first length and the low density portions 75 a second different length. Advantageously, the pattern of varying lengths can help optimize the echogenicity. For example, the pattern of varying lengths may help to balance the brightness relative to artifacts in the imaging, which can help distinguish different portions of the anchor 15 (e.g., an inner portion of the anchor 15 relative to an outer portion of the anchor 15), or mark specific points on the anchor 15 for tracking during imaging.

In some embodiments, the echogenic portions (e.g., portions 71, 22) of the anchor 15 can include a patterned or textured surface treatment (e.g., configured to disperse ultrasonic waves in a plurality of directions) in addition to or in place of the coil 73. In some embodiments, the echogenic portions (e.g., portions 71, 72) of the anchor 15 can include a fabric or a polymer wrapped or layered therearound in addition to or in place of the coil 73.

In some embodiments, the echogenic portions (e.g., portions 71, 22) of the anchor 15 can be configured to be clearly visible under fluoroscopy in addition to ultrasound.

FIG. 8 shows a variation of the anchor of FIGS. 7A and 7B where the echogenic portions 71 along the main body 18 include a plurality of echogenic markers 77. In some embodiments, the markers 77 are or include bands that wrap around the central component (e.g., central wire) of the anchor. The echogenic markers 77 may be similar to the high-density and low-density portions 74, 75 in FIGS. 7A and 7B and/or the sections of varying length otherwise described herein that extend along the length of the main body 18 (and/or grabber arm 91). For example, the markers 77 may have predetermined axial lengths (e.g., equal or unequal axial lengths), have predetermined shapes (e.g., the same or different shapes), and spaced apart by predetermined distances (e.g., equal or unequal spacings). Such arrangement of echogenic markers 77 can be used to provide an indication as to the planarity of the anchor 15 during delivery. For example, such predetermined arrangement of markers 77 can allow visualization of distinct sections of the anchor 15, which can be used to indicate the angular position of the anchor 15 when viewed under ultrasound. That is, such lengths, shapes and/or spacings can provide a visible signature of a particular portion (or multiple positions) of the anchor 15 during placement of the anchor 15.

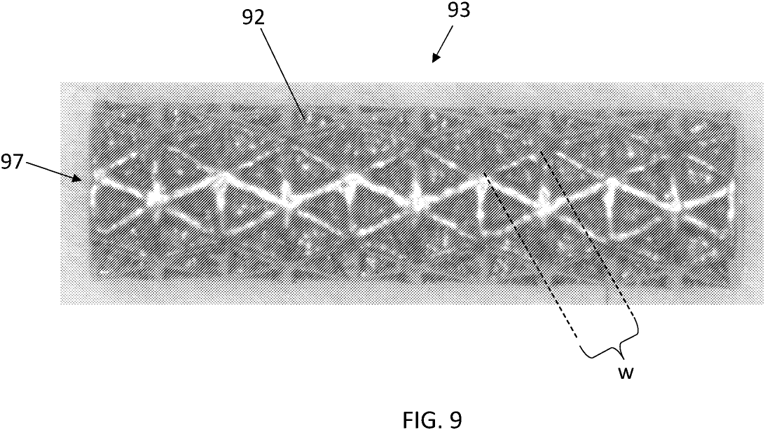

FIG. 9 illustrates an exemplary echogenic portion 93 that is a multifaceted feature. The echogenic portion 93 can have a generally tubular profile with an interior lumen 97 for accepting and coupling with a portion of the anchor. For example, the interior lumen 97 may be sized and shaped to accept and couple with a wire portion at the distal end of the grabber arm 91. The echogenic portion 93 includes multiple reflective elements 92 that are configured to reflect ultrasound waves. In some examples, the reflective elements 92 each have a width ranging from about 50 micrometers (µm) and about 300 µm. In some examples, the reflective elements 92 each have a width “w” on each side ranging from about 100 µm and about 200 µm. In some examples, the reflective elements 92 each have a depth ranging from about 10 µm and about 100 µm.

In some examples, the reflective elements 92 may each be a retroreflector, i.e., configured to reflect waves (e.g., ultrasound) back in the direction toward the source of the waves. The reflective elements 92 may each include an arrangement of planar reflective surfaces that form an inset (also referred to as a recess) with respect to an outer surface of the echogenic portion 93. The reflective surfaces may have any of a number of geometric shapes (e.g., triangles, squares, rectangles, pentagons, hexagons, octagons and/or circles). In the example of FIG. 9, the reflective elements 92 are cubic reflectors (also referred to as cube-corner reflectors) that each include three perpendicularly oriented reflective surfaces. Other examples of suitable retroreflectors may include a cat’s eye retroreflector and/or a spherical retroreflector.

In some embodiments, the reflective elements 92 are arranged entirely or partially around a diameter of the echogenic portion 93 (e.g., the distal tip of the anchor). In some embodiments, the reflective elements 92 are arranged in a pattern (e.g., repeated pattern) along the echogenic portion 93 where some regions of the echogenic portion 93 include reflective elements 92 and other regions of the echogenic portion 93 do not include reflective elements 92.

In some cases, at least a portion of the echogenic portion 93 is made of a metal material. In some examples, the metal material may include stainless steel, nitinol, or stainless steel and nitinol. In some cases, at least a portion of the echogenic portion 93 is made of the same material as at least a portion of the main body (e.g., 18) and/or the grabber arm (e.g., 91) of the anchor. In some cases, the echogenic portion 93 is encapsulated in one or more layers of non-conductive and/or non-metallic material, such as described herein with respect to FIG. 3A. In one example, once the echogenic portion 93 is inserted over the wire portion of the anchor (e.g., grabber arm 91 or main body 18), the main body 18, the grabber arm 91 and/or the echogenic portion 93 may be laminated to provide an outer covering to the anchor.

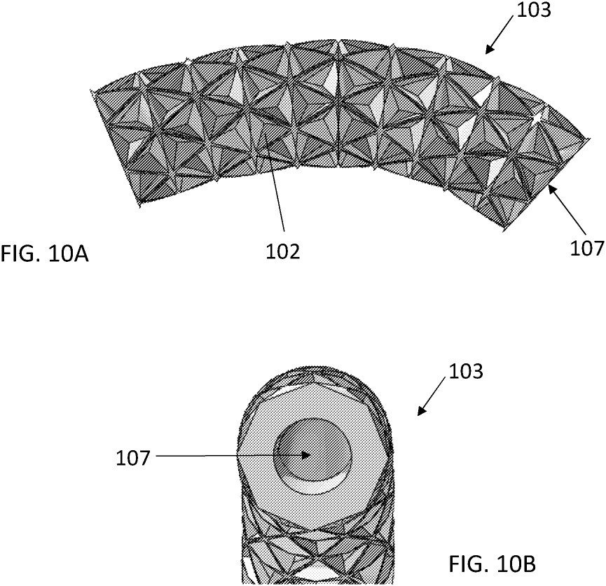

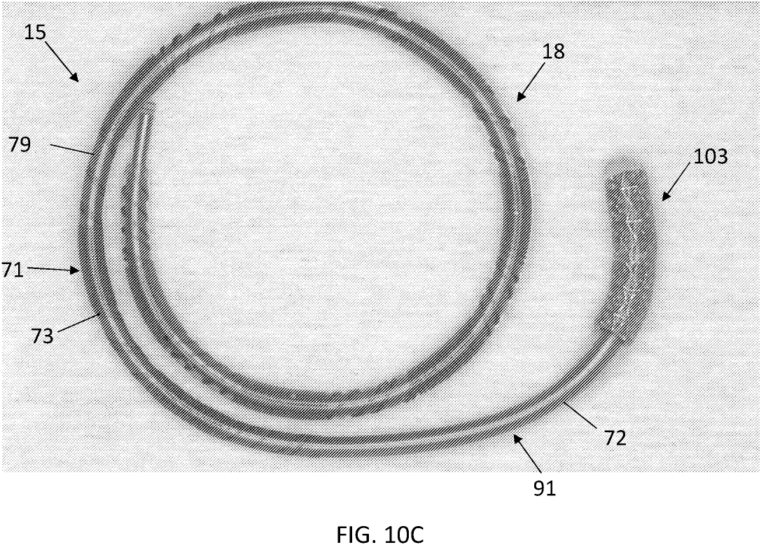

FIGS. 10A-10C show an exemplary variation of the echogenic portion 93 of FIG. 9. The echogenic portion 103 includes an arrangement of multiple reflective elements 102 (e.g., cubic reflectors), like the echogenic portion 93 of FIG. 9. However, in this case, the echogenic portion 103 has a curved geometry (e.g., compared to a straight or linear geometry of the echogenic portion 93 of FIG. 9). When coupled to the rest of the anchor, the curvature of the echogenic portion 103 can be in a same direction as a curvature of the main body 18 and the grabber arm 91 of the anchor 15, as shown in FIG. 10C. This curvature may facilitate in maneuvering of the anchor 15 within the heart and wrapping of the anchor 15 around heart tissue (e.g., chordae).

As shown in FIG. 10B, the multifaceted feature 103 can include an interior lumen 106 that is sized and shape to accept a wire portion at the distal end of the grabber arm 91. As with the echogenic portion 93 of FIG. 9, the echogenic portion 103 may be laminated (e.g., with the wire portions of the grabber arm 91 and/or main body 18) to provide an outer covering to the anchor 15.

Returning to FIG. 10C, in this example the anchor 15 includes additionally includes echogenic portions 71 (as part of the main body 18) and a non-echogenic portion 72 (corresponding to the grabber arm 91), similar to the example of FIGS. 7A-7C. In some embodiments, the echogenic portions 71 include an echogenic wire or coil 73 (e.g., made of stainless steel and/or nitinol) that wraps around central component 79 (e.g., central wire) of the main body 18. The wire/coil can be wrapped in a predetermined pattern (e.g., high density and/or low density patterns) along at least a portion of the length of the anchor 15 that allows for visual distinction of different regions of the main body 18 when view by ultrasound, and which may allow for improved determination of the orientation of the anchor 15, as described herein. In other embodiments, the pattern(s) of echogenic portions along the main body 18 may be, or include, markers (e.g., bands), such as described with reference to FIG. 8.

Any of the anchors described herein can include echogenic and non-echogenic portions in arrangements other that as shown in FIGS. 7A-10C. For example, in some embodiments, all or a portion of the grabber arm (e.g., 91) may be echogenic and the main body (e.g., 18) may be non-echogenic. For example, in some embodiments, the coil (e.g., 73), markers (e.g., 77) and/or reflective elements (e.g., 92) may extend along all or a portion of the length of the main body (e.g., 18) but not within the grabber arm (e.g., 91). In some embodiments, all or a portion of the main body (e.g., 18) and the grabber arm (e.g., 91) may be echogenic. For example, in some embodiments, the coil (e.g., 73) markers (e.g., 77) and/or reflective elements (e.g., 92) may extend along all or a portion of the lengths of both the main body (e.g., 18) and the grabber arm (e.g., 91). In some embodiments, all or a portion of the main body (e.g., 18) the grabber arm (e.g., 91) may be non-echogenic. For example, in some embodiments, neither the main body (e.g., 18) nor the grabber arm (e.g., 91) may include the coil 73 markers (e.g., 77) or reflective elements (e.g., 92).

In general, the echogenic portion at the distal tip of any of the anchors described herein (e.g., coiled or multifaceted) may vary in axial length and diameter. The length of the echogenic portion may be long enough to provide easy detection via ultrasound imaging yet short enough to allow most of the main body of the anchor to readily transition from the elongated undeployed state to the spiral-shaped deployed state. In some examples, the length of the echogenic portion ranges between any two of the following values: 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, and 10 mm. The diameter of the echogenic portion may be large enough to provide easy detection via ultrasound imaging yet small enough to be stored within a catheter (e.g., of an anchor delivery catheter system) prior to deployment. In some examples, the diameter of the echogenic portion is greater than the diameter of the main body (e.g., proximal portion 71 and/or distal portion 72). In some examples, the diameter of the echogenic portion ranges between any two of the following values: 2 mm, 2.5 mm, 3 mm, 3.5 mm, and 4 mm.

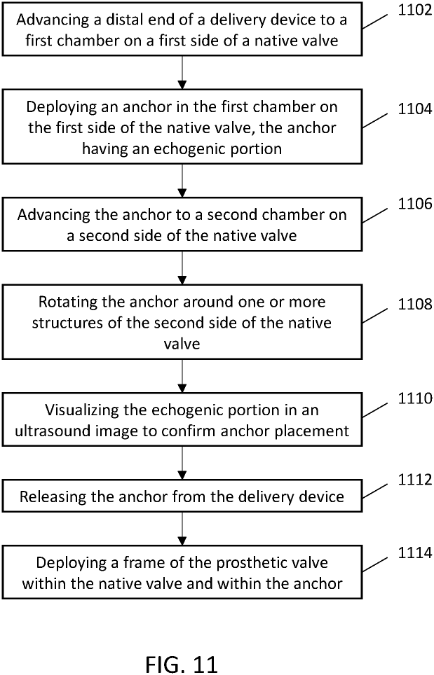

FIG. 11 is a flowchart indicating an exemplary method of delivering an anchor and a valve prosthesis into a patient’s heart. At 1102, a distal end of a delivery device is advanced to a first chamber on a first side (e.g., left atrium side) of a native valve. At 1104, an anchor is deployed from a delivery configuration to a deployed configuration within the first chamber. In some cases, the anchor is deployed from a delivery device (e.g., anchor delivery catheter system). The anchor can include one or more echogenic portions, as described herein. For example, the anchor may include an echogenic distal tip and/or one or more echogenic regions along a main body and/or a grabber arm of the anchor. At 1106, the anchor is advanced in the deployed configuration from the first side of the native valve to a second chamber on a second side (e.g., left ventricle) of the native valve. At 1108, the anchor is rotated while the deployed configuration around one or more structures on the second side of the native valve. For example, the anchor may be rotated around the chordae and/or leaflets of the native valve. Rotating the anchor may include capturing the one or more structures on the second side of the heart using with an outwardly radially extending grabber arm of the anchor. At 1110, the one or more echogenic portions are visualized in an ultrasound image to confirm that the anchor has been fully rotated around the one or more structures. In some cases, the echogenic portion may additionally or alternatively be visualized by using ultrasound imaging during one or more procedures during placement of the anchor, e.g., during advancement of the anchor (e.g., at 1106) and/or during rotation of the anchor (e.g., at 1108). In some examples, visualizing the echogenic portion in the ultrasound image includes confirming an orientation of the anchor (e.g., with respect to the native valve). For example, it may be desirable for the anchor to be in a substantially planar configuration with respect to the native valve. In some cases, the anchor includes more than one echogenic portion (e.g., at the distal tip and/or one or more of the main body and/or the grabber arm) to facilitate identification of the orientation of the anchor. At 1112, the anchor is released from a distal end of the delivery device. At 1114, a frame of the valve prosthesis is deployed within the native valve and within the anchor.

Additional elements of valve prostheses, anchors, and methods of delivery are described in PCT Application No. PCT/US2019/047542 filed on Aug. 21, 2019, PCT Application No. PCT/US2019/057082 filed on Mar. 19, 2019, PCT Application No. PCT/US2019/068088 filed on Dec. 20, 2019, and PCT Application No. PCT/US2020/023671, the entireties of which are incorporated herein by reference.

It should be understood that any feature described herein with respect to one embodiment can be substituted for or combined with any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/- 0.1% of the stated value (or range of values), +/- 1% of the stated value (or range of values), +/- 2% of the stated value (or range of values), +/- 5% of the stated value (or range of values), +/- 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-30. (canceled)

31. A method of delivering an anchor of a valve prosthesis, the method comprising:
advancing a distal end of a delivery device to a first side of a native valve;
deploying an anchor from a delivery configuration to a deployed configuration on the first side of the native valve, the anchor comprising an echogenic portion;
advancing the anchor in the deployed configuration from the first side of the native valve to a second side of the native valve;
rotating the anchor in the deployed configuration around one or more structures on the second side of the native valve;
identifying the echogenic portion in an ultrasound image to confirm that the anchor has been fully rotated around the one or more structures; and
releasing the anchor from the distal end of the delivery device.

32. The method of claim 31, further comprising identifying the echogenic portion with ultrasound during the rotating step.

33. The method of claim 31, further comprising identifying the echogenic portion with ultrasound during the advancing step.

34. The method of claim 31, wherein identify the echogenic portion in the ultrasound further comprises confirming a planarity of the anchor.

35. (canceled)

36. The method of claim 31, further comprising deploying a frame of the valve prosthesis within the native valve and within the anchor.

37. The method of claim 31, further comprising capturing the one or more structures on the second side of the heart using with an outwardly radially extending grabber arm of the anchor.

38. The method of claim 31, wherein the anchor has a spiral shape.

39. The method of claim 31, wherein the echogenic portion is at least a distal tip of the anchor, wherein identifying the echogenic portion includes identifying a location of at least the distal tip of the anchor.

40. The method of claim 31, wherein the echogenic portion is at least a portion of a main body of the anchor, the main body having a spiral shape, wherein identifying the echogenic portion includes identifying a location of at least the portion of the main body of the anchor.

41. The method claim 31, wherein the echogenic portion is at least a portion of an outwardly radially extending grabber arm of the anchor, wherein identifying the echogenic portion includes identifying a location of at least the portion of the grabber arm of the anchor.

42. The method of claim 31, wherein the echogenic portion comprises a wire wound around the distal tip.

43. The method of claim 31, wherein the echogenic portion comprises reflective elements each having an arrangement of reflective surfaces that reflect ultrasound.

44. The method of claim 31, further comprising releasing the anchor from the delivery device after anchor placement is confirmed.

45-63. (canceled)
